(19) United States
(12) Patent Application Publication
(10) Pub. No.: US 2010/0080777 A1
(43) Pub. Date: Apr. 1, 2010

(54) COMPOSITION CONTAINING AUTOLOGOUS MONONUCLEAR CELLS AND PIG COLLAGEN SCAFFOLD IN PASTE FORM AND ITS USE IN THE PREPARATION OF A MEDICAMENT FOR SURGICAL TREATMENT

(75) Inventors: Sandro Giannini, Viareggio (IT); Roberto Buda, Sasso Marconi (IT); Brunella Grigolo, Bologna (IT)

Correspondence Address: LUCAS & MERCANTI, LLP, 475 PARK AVENUE SOUTH, 15TH FLOOR, NEW YORK, NY, 10016, US

(73) Assignee: ISTITUTO ORTOPEDICO RIZZOLI, Bologna (IT)

(21) Appl. No.: 12/530,081

(22) PCT Filed: Mar. 7, 2008
(86) PCT No.: PCT/IB08/00530

§ 371 Date: Sep. 4, 2009

(30) Foreign Application Priority Data
Mar. 7, 2007 (IT) MI2007A000458

(51) Int. Cl. A61K 35/12; C12N 5/02

(52) U.S. Cl. 424/93.7; 435/395

(57) ABSTRACT

Composition including autologous mononuclear cells supported on scaffold chosen from the group including lyophilized scaffold or pre-constituted scaffold in the form of membrane or tissue, with possible addition of platelet gel, as well as use of said composition for the preparation of medicaments for surgical treatments.

### TECHNICAL FIELD

The present invention concerns the treatment of osteochondral articular lesions.

Purpose of the repair of the articular cartilage is to restore the integrity of the joint surface, through the regeneration of hyaline cartilage. Hyaline cartilage can be obtained by methods that employ cell transplantations of chondrocytes (or their precursors).

### BACKGROUND ART

Nowadays, the state of the art involves the implant of autologous chondrocytes by arthroscopy. Such method entails a preliminary arthroscopy to harvest cartilage from the areas adjacent to the lesion or from a detached chondral fragment; the harvested chondrocytes are expanded in in vitro cell culture for a mean period of 5 weeks and seeded on biocompatible and bioabsorbable scaffolds based on hyaluronic acid, collagen or others; in a second arthroscopy the chondrocytes are implanted on scaffolds. This technique has the drawback of requiring two surgical sessions, the time for the necessary phase of in vitro cell expansion and the preparation of environments suitable for this purpose, high costs for the whole procedure, and great inconvenience for the patient. The characterizing limit of the employment of chondrocytes is the fact that they cannot be isolated if not in small quantities from cartilaginous tissue, and they require therefore a quantitative expansion in the laboratory. Such a process takes at least 15-20 days, and does not allow therefore the execution of a “one step” surgical technique.

Therefore, the need was particularly felt to overcome the drawbacks in terms of time, costs and inconvenience for the patient that the technique of transplantation of autologous chondrocytes involves.

### DISCLOSURE OF THE INVENTION

The applicants have surprisingly conceived and realized a biological material or composition including autologous mononuclear cells, more precisely, autologous mononuclear cells isolated from bone marrow, from peripheral blood or from fat tissue, supported by scaffold chosen from the group including lyophilized scaffold or pre-constituted scaffold in the form of membrane or tissue, with possible addition of platelet gel or other different signalling molecules. Said composition has been conceived for the preparation of medicaments for the-treatment of osteocartilaginous articular lesions to be used in arthroscopic or arthrotomic surgery, in a single operating session.

According to the present invention it is possible to perform the full treatment in a single operating session, contrarily to the methods previously used that involve two surgical sessions.

Autologous mononuclear cells, according to the present invention, in fact, unlike chondrocytes, can be harvested in great quantities and packed, directly during the operating session.

In the orthopedic field its usefulness is known in the treatment of nonunions (Hemigou, et al.: J Bone Joint Surg Br 2005 Jul;87 (7): 896-902) and necroses of the femoral head (Gangji, et al.: J Bone Joint Surg Am. 2004 Jun;86-A (6): 1153-60). To be implanted in great number in the lesion area, such cells need a scaffold on which to be seeded.

The scaffolds that belong to the composition of the present invention, can be lyophilized scaffolds, particularly collagen of pig origin (such as Spongostan® Powder or Surgiflo®). This is a bioabsorbable and biocompatible material already broadly used in surgery to provide surgical haemostasis, which acts well as a malleable scaffold to deliver the cells to the site of the lesion, but never indicated as possible scaffold to be combined with autologous mononuclear cells, preferably those isolated from bone marrow, peripheral blood or fat.

The composition of the present invention, besides its employment in the treatment of osteocartilaginous articular injuries in a single operating session in arthroscopic or arthrotomic surgery, allows the aforementioned drawbacks of the autologous chondrocyte transplantation technique to be overcome.

One object of the present invention consists of a biological material or composition including autologous mononuclear cells supported on scaffold chosen from the group including lyophilized scaffold or pre-constituted scaffold in the form of membrane or tissue.

In a preferred form of realization of the composition of the present invention, the autologous mononuclear cells used are autologous mononuclear cells of medullary origin, particularly those harvested from the posterior iliac crest, or autologous mononuclear cells isolated from peripheral blood or from fat.

In a further even more preferred form of realization of the composition according to the invention, the scaffold supporting the autologous mononuclear cells, more precisely those of medullary origin, chosen from the group including lyophilized scaffold or pre-constituted scaffold in the form of membrane or tissue, is a biocompatible and bioabsorbable scaffold and can be constituted by various composite materials, such as natural, semi-synthetic or synthetic polymers.

The natural polymers used for the invention are selected from the group including collagen, collagen and glycosaminoglycan co-precipitates, hyaluronic acid, cellulose, polysaccharides in the form of chitin gel, chitosan, pectin or pectic acid, agar, agarose, xanthan, gellan gum, alginic acid or alginates, polymannans or polyglycans, starch and natural rubbers, and demineralized bone matrix (DBM).

The semi-synthetic polymers used for the invention are selected from the group including reticulated collagen with agents such as aldehydes or their precursors, dicarboxylic acids or their halides, diammine, derivates of cellulose, hyaluronic acid, chitin gel or chitosan, gellan gum, xanthan, pectin or pectic acid, polyglycans, polymannans, agar, agarose, natural rubber and glycosaminoglycan.

The useful synthetic polymers to achieve the invention are selected from the group including polylactic acid, polyglycolic acid or their copolymers or derivates, polydioxane, polyphosphazenes, polysulfonic resins, polyurethanes and PTFE, hydroxyapatite in paste form.

In a preferred realization of the invention the composition includes a scaffold constituted by pig collagen.

In addition, it has been found that various signalling molecules, including growth factors, can be added to the composition of the present invention to favor the multiplication and the differentiation of the autologous mononuclear cells, particularly those from packed bone marrow.

In a further preferred form of realization of the composition of the present invention, these growth factorsare provided platelet gel is obtained by harvesting venous blood the day before the operation, through an automated procedure, such as Vivostat®.

The platelet gel according to the invention can be placed on the surface of the biological material or composition of the present invention or on the bed of the lesion or in the composition of the paste.

The biological material or composition of the present invention can also include pharmacologically or biologically active substances. These pharmacologically and biologically active substances are selected from the group including anti-inflammatory agents, antibiotics, growth factors, and antimycotic and antiviral agents.

Another object of the present invention is the use of biological material or composition, in any of the forms described above, for the preparation of a medicament for the treatment of articular osteocartilaginous injuries in a single operating session in arthroscopic or arthrotomic surgery.

In a preferred form of realization of the present invention articular osteocartilaginous lesions are ostoarticular lesions of knees and ankles.

According to the present invention it has also been found that it is possible to preserve, in a preferred form cryopreserve, preventively the biological material or composition, according to any of the forms of preferred realization described above, to store its characteristics of cellular viability for grafts in subsequent arthroscopic or arthrotomic procedures.

Another object of the present invention is a method of treatment of articular osteocartilaginous lesions in a single operating session by the implant in arthroscopic or arthrotomic surgery of a biological material or composition including autologous mononuclear cells and a scaffold chosen by the group including lyophilized scaffold or pre-constituted scaffold in the form of membrane or tissue, said method including:
Drawing of medullary blood from the posterior iliac crest during the operating session;
Preparation of a concentrate of autologous mononuclear cells from medullary aspirate with an automated method of centrifugation in the operating room, (such as the BMAC Harvest® method or others);
Arthroscopy or arthrotomy of the joint to be treated;
Arthroscopic or arthrotomic cleaning of the lesion and relief of combined conditions;
Preparation of a composition including the concentration of autologous mononuclear cells from medullary aspirate and scaffold of support chosen by the group including lyophilized scaffold or pre-constituted scaffold in the form of membrane, said composition obtained for mixing said components together;
Implant of the composition to fill the center of infection of the osteochondral lesion in arthroscopy or arthrotomy.

In a preferred form of the method of treatment of articular osteocartilaginous injuries in a single operating session according to the present invention, the lyophilized scaffold is pig collagen (such as Spongostan® Powder or Surgiflo®.

In a further preferred form of realization of the method of treatment of articular osteocartilaginous injuries in a single operating session according to the present invention, said method also includes before the phase of drawing of the medullary blood from the iliac crest the following phases:
Drawing of venous blood the day before the operation;
Preparation of platelet gel with automated procedure, such as the Vivostat® procedure;
freezing of the platelet gel up to the moment of the operation; from which the preserved platelet gel is, alternatively:
1) mixed in the composition including the concentration of autologous mononuclear cells from medullary aspirate and support scaffold; or
2) placed in the interface between the composition including the concentration of autologous mononuclear cells from medullary aspirate and support scaffold and the bed of the osteo-chondral lesion; or
3) spread on the implant of the composition including the concentration of autologous mononuclear cells from medullary aspirate and support scaffold, the implant in turn is spread on the center of infection of the osteoarticular lesion; or
4) mixed in the composition according to point 1) and situated in the interface according to point 2); or
5) mixed in the composition according to point 1) and spread on the implant according to point 3); or
6) placed on the interface according to point 2) and spread on the implant according to point 3); or
7) contemporarily present in the composition according to point 1), placed on the interface according to point 2) and spread on the implant according to point 3).

The rationale of the method of treatment of articular osteocartilaginous injuries in a single operating session, according to the present invention, includes as a further preferred form:
the drawing of venous blood the day before the operation for the preparation of the platelet gel with automated method, such as Vivostat® with possible freezing of the obtained platelet gel (these phases in optional form);
in the operating site a medullary aspirate from the posterior iliac crest is performed, it is concentrated in the operating room by an automated method of centrifugation, with the use for instance of the Harvest device to get a concentration of mononuclear cells;
during the concentration of the cells an arthroscopy or arthrotomy is performed of the joint to be treated with relief and recovery of the osteochondral lesion;
the medullary concentrate is used for preparing a composition, preferably in paste form, by mixing with the collagen of pig origin (such as Spongostan® Powder or surgiflo®);
the composition or paste is subsequently inserted by arthroscopy or arthrotomy to fill the site of the lesion;
finally, optionally, a layer of platelet gel is spread onto it or, alternatively, the platelet gel can be placed on the bed of the lesion or in the composition or paste when the prepared platelet gel is used in the optional phases.

A further object of the present invention includes the process for the preparation of the composition, in any of the preferred foams of realization described above, said process includes:
Preparation of a concentration of autologous mononuclear cells from medullary aspirate with an automated method of centrifugation in the operating room, such as the BMAC® Harvest method;
Preparation of an composition including the concentrate of autologous mononuclear cells from medullary aspirate and support scaffold chosen by the group including lyophilized scaffold or pre-constituted scaffold in the form of membrane or tissue, said composition obtained for mixing said components together.

Optionally said process of preparation includes in the phase of preparation of the composition the mixing of platelet gel obtained by the automated procedure with venous blood, or having obtained the composition of autologous mononuclear cells and scaffold, the distribution around the said composition, in contact with it, of platelet gel, preferably in layer form, obtained with the automated procedure from venous blood.

1. Composition including autologous mononuclear cells supported by scaffold chosen from the group including lyophilized scaffold or pre-constituted scaffold in the form of membrane or tissue.

2. Composition according to claim 1, in which the autologous mononuclear cells are autologous mononuclear cells of medullary origin or autologous mononuclear cells isolated from peripheral blood or autologous mononuclear cells isolated from fat.

3. Composition according to claim 2 in which the autologous mononuclear cells are of medullary origin.

4. Composition according to claim 3, in which the autologous mononuclear cells of medullary origin are withdrawn from the posterior iliac crest.

5. Composition according to claim 1, in which the lyophilized scaffold is constituted by pig collagen.

6. Composition according to claim 1, in which the scaffold chosen from the group including lyophilized scaffold or pre-constituted scaffold in the faun of membrane or tissue is constituted by natural polymers, or semi-synthetic polymers, or synthetic polymers.

7. Composition according to claim 6, in which the natural polymers are selected from the group including collagen, collagen and glycosaminoglycan co-precipitates, hyaluronic acid, cellulose, polysaccharides in the form of chitin gel, chitosan, pectin or pectic acid, agar, agarose, xanthan, gellan gum, alginic acid or alginates, polymannans or polyglycans, starch and natural rubbers, and demineralized bone matrix (DBM).

8. Composition according to claim 6 in which the semi-synthetic polymers are selected from the group including reticulated collagen with agents such as aldehydes or their precursors, dicarboxylic acids or their halides, diammine, derivates of cellulose, hyaluronic acid, chitin gel or chitosan, gellan gum, xanthan, pectin or pectic acid, polyglycans, polymannans, agar, agarose, natural rubber and glycosaminoglycan.

9. Composition according to claim 6, in which the synthetic polymers are selected from the group including polylactic acid, polyglycolic acid or their copolymers or derivates, polydioxane, polyphosphazenes, polysulfonic resins, polyurethanes and PIM, hydroxyapatite in paste or tissue form.

10. Composition according to claim 1, also including a platelet gel.

11. Composition according to claim 1, also including pharmacologically or biologically active substances.

12. Composition according to claim 11, in which the pharmacologically and biologically active substances are selected from the group including anti-inflammatory agents, antibiotic, growth factors, antimycotic and antiviral agents.

13. Composition according to claims 1-12, in which the composition is preserved for subsequent grafts.

14. Composition according to claims 1-12, in which the composition is cryopreserved.

15. Composition according to claims 1-14 for use as a medicament.

16. Use of a composition according to claims 1-14, for the preparation of a medicament for the treatment of articular osteocartilaginous injuries in arthroscopic surgery in a single operating session.

17. Use of a composition according to claims 1-14, for the preparation of a medicament for the treatment of articular osteocartilaginous injuries in arthrotomic surgery in a single operating session.

18. Process for the preparation of a composition including autologous mononuclear cells of medullary origin supported by scaffold chosen from the group including lyophilized scaffold or pre-constituted scaffold in the form of membrane or tissue of the composition, composition optionally including also platelet gel, including said process:
Preparation of a concentrate of autologous mononuclear cells from medullary aspirate by automated method of centrifugation in the operating room;
Preparation of an composition including the concentrate of autologous mononuclear cells from medullary aspirate and support scaffold chosen from the group including lyophilized scaffold or pre-constituted scaffold in the form of membrane or tissue, said composition obtained for mixing said components together;
optionally, said process of preparation includes:
in the phase of preparation of the composition the mixing also of platelet gel obtained by automated procedure with venous blood or
having obtained the composition of mononuclear cells and scaffold, distribution around said composition, in contact with it, of platelet gel, preferably in layer form, by obtained automated procedure with venous blood.
